(19) United States
(12) Patent Application Publication
(10) Pub. No.: US 2024/0245445 A1
(43) Pub. Date: Jul. 25, 2024

(54) RF ABLATION SYSTEMS WITH INTEGRATED FLUID DELIVERY AND METHODS FOR MAKING AND USING

(71) Applicant: Boston Scientific Neuromodulation Corporation, Valencia, CA (US)

(72) Inventor: Gregory Bates, Bend, OR (US)

(21) Appl. No.: 18/414,815

(22) Filed: Jan. 17, 2024

Related U.S. Application Data
(60) Provisional application No. 63/440,612, filed on Jan. 23, 2023.

(51) Int. Cl. A61B 18/14; A61B 18/00; A61B 18/12

(52) U.S. Cl. CPC A61B 18/14; A61B 2018/00083; A61B 2018/00166; A61B 2018/00178; A61B 2018/00577; A61B 2018/00964; A61B 2018/126; A61B 2018/1467; A61B 2218/002

(57) ABSTRACT

A bipolar RF electrode includes an electrode shaft having a first end portion and a second end portion opposite the first end portion; a first electrode; a second electrode attached to the second end portion of the electrode shaft; an insulative material coupled to, and disposed between, the first electrode and the second electrode, the insulative material defining at least one fluid delivery port; and an electrode hub attached to first end portion of the electrode shaft, wherein the electrode hub or the electrode shaft is configured for attachment of a fluid line, wherein at least the electrode shaft, the second electrode, and the insulative material form a hollow interior for flow of fluid from the fluid line, when attached, to the at least one fluid delivery port defined by the insulative material and disposed between the first electrode and the second electrode.

### CROSS-REFERENCE TO RELATED APPLICATIONS

This application claims the benefit under 35 U.S.C. § 119(e) of U.S. Provisional Patent Application Ser. No. 63/440,612, filed Jan. 23, 2023, which is incorporated herein by reference.

### FIELD

The present disclosure is directed to the area of radiofrequency (RF) ablation systems and methods of making and using the systems. The present disclosure is also directed to RF ablation systems and methods that include integrated fluid delivery, as well as methods of making and using the same.

### BACKGROUND

Radiofrequency (RF) generators and electrodes can be used for pain relief or functional modification. Radiofrequency ablation (RFA) is a safe, proven means of interrupting pain signals, such as those coming from irritated facet joints in the spine, genicular nerves in the knee, and femoral and obturator nerves in the hip. Radiofrequency current is used to heat up a small volume of nerve tissue, thereby interrupting pain signals from that specific area. Radiofrequency ablation is designed to provide long-lasting pain relief.

For example, an RF electrode can be positioned near target tissue and then used to heat the target tissue by RF power dissipation of the RF signal output in the target tissue. Temperature monitoring of the target tissue by a temperature sensor in the electrode may be used to control the process.

### BRIEF SUMMARY

One aspect is a bipolar RF electrode that includes an electrode shaft having a first end portion and a second end portion opposite the first end portion; a first electrode; a second electrode attached to the second end portion of the electrode shaft; an insulative material coupled to, and disposed between, the first electrode and the second electrode, the insulative material defining at least one fluid delivery port; and an electrode hub attached to first end portion of the electrode shaft, wherein the electrode hub or the electrode shaft is configured for attachment of a fluid line, wherein at least the electrode shaft, the second electrode, and the insulative material form a hollow interior for flow of fluid from the fluid line, when attached, to the at least one fluid delivery port defined by the insulative material and disposed between the first electrode and the second electrode.

In at least some aspects, the at least one fluid delivery port is a plurality of fluid delivery ports disposed around a circumference of the bipolar RF electrode. In at least some aspects, the electrode hub is configured for attachment of the fluid line. In at least some aspects, the electrode shaft is configured for attachment of the fluid line.

In at least some aspects, the bipolar RF electrode further includes a cable extending from the electrode hub and a plurality of conductors extending along the cable and the electrode shaft, wherein at least one conductor is electrically coupled to the first electrode and at least one other conductor is electrically coupled to the second electrode.

In at least some aspects, the first electrode has a closed end. In at least some aspects, the insulative material is part of the electrode shaft. In at least some aspects, the second electrode is disposed over a portion of the electrode shaft.

Another aspect is a kit that includes any of the bipolar RF electrodes described above and a cannula configured for insertion of the electrode shaft through the cannula. In at least some aspects, the kit further includes the fluid line.

A further aspect is a RF ablation system that includes any of the bipolar RF electrodes described above; a cannula configured for insertion of the electrode shaft through the cannula; and a RF generator configured for electrically coupling to the bipolar RF electrode and energizing at least one of the first electrode or the second electrode.

In at least some aspects, the RF ablation system further includes the fluid line. In at least some aspects, the RF ablation system further includes a fluid source coupleable to the fluid line for providing fluid to the fluid line for delivery through the bipolar RF electrode and out the at least one fluid delivery port. In at least some aspects, the fluid includes a numbing agent. In at least some aspects, the fluid includes a contrast agent. In at least some aspects, the fluid includes a conductive liquid to enhance ablation. In at least some aspects, the fluid includes a healing medication. In at least some aspects, the fluid includes embolic beads configured to starve tissue of blood flow.

Yet another aspect is a method for performing RF ablation. The method includes positioning the first and second electrodes of any of the bipolar RF electrodes described above proximate to an ablation target of a patient; delivering fluid from a fluid source through a fluid line and the bipolar RF electrode coupled to the fluid line and out the at least one fluid delivery port defined by the insulative material of the bipolar RF electrode; and ablating tissue using the first and second electrodes of the bipolar RF electrode.

In at least some aspects, the fluid includes a numbing agent. In at least some aspects, the method further includes confirming the positioning of the first and second electrodes by numbing of the patient by the numbing agent.

In at least some aspects, the fluid includes a contrast agent, the method further including imaging the ablation target and first and second electrodes after delivery of the contrast agent. In at least some aspects, the fluid includes a conductive liquid to enhance ablation. In at least some aspects, the fluid includes a healing medication, wherein the delivering occurs after the ablating. In at least some aspects, the fluid includes embolic beads configured to starve tissue of blood flow.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1 is a schematic side view of components of one embodiment of a RF ablation system with a bipolar RF electrode;

FIG. 2 is a schematic perspective view of a distal portion of one embodiment of a bipolar RF electrode and cannula, where the bipolar RF electrode includes at least one fluid delivery port;

FIG. 3 is a schematic diagram of another embodiment of a bipolar RF electrode and cannula with a fluid line and connector coupled to the bipolar RF electrode for delivery of fluid through the at least one fluid delivery port in the RF bipolar electrode; and

FIG. 4 is a schematic side view of components of one embodiment of an adapter for coupling a bipolar RF electrode to a RF generator.

### DETAILED DESCRIPTION

The present disclosure is directed to the area of radiofrequency (RF) ablation systems and methods of making and using the systems. The present disclosure is also directed to RF ablation systems and methods that include integrated fluid delivery, as well as methods of making and using the same.

The basivertebral nerve (BVN) is located at the center of vertebrae in the lower back. The BVN can be difficult to access. To improve effectiveness, the ablation volume of the nerve should be large enough to eliminate the pain and prevent the nerve from growing back quickly. The location of the BVN can vary in vertebrae. Placement of the ablation electrode(s) may not provide full ablation due to the variation in the location of the BVN. This may decrease the therapy effectiveness. Furthermore, the patient may experience pain after the procedure due to trauma inside the vertebrae caused by the sharp access tools and the ablation itself.

As described herein, a bipolar RF electrode can be constructed to allow for fluid flow between the two electrodes for delivery of fluid, drugs, medications, contrast agents, or the like through the bipolar RF electrode and directly to the ablation site. In contrast, fluid or drugs delivered through the cannula used to insert the bipolar RF electrode would likely be delivered at a site that is a significant distance (for example, 15 to 40 mm or more) away from the ablation site. The response or effect of the fluid delivery through the cannula to the ablation site can be inconsistent or unpredictable due to this distance.

One electrode supplies power while the other electrode acts as a return. Each electrode requires one channel on the RF generator.

As described herein, a RF ablation system can include a bipolar RF (radiofrequency) electrode (i.e., a component with two electrodes on the same shaft), instead of two or more monopolar electrodes. In at least some embodiments, one electrode supplies power while the other electrode acts as a return. Each electrode requires one channel of the RF generator. In at least some embodiments, an RF generator that was previously used for monopolar electrodes can be used or adapted for use with a bipolar RF electrode.

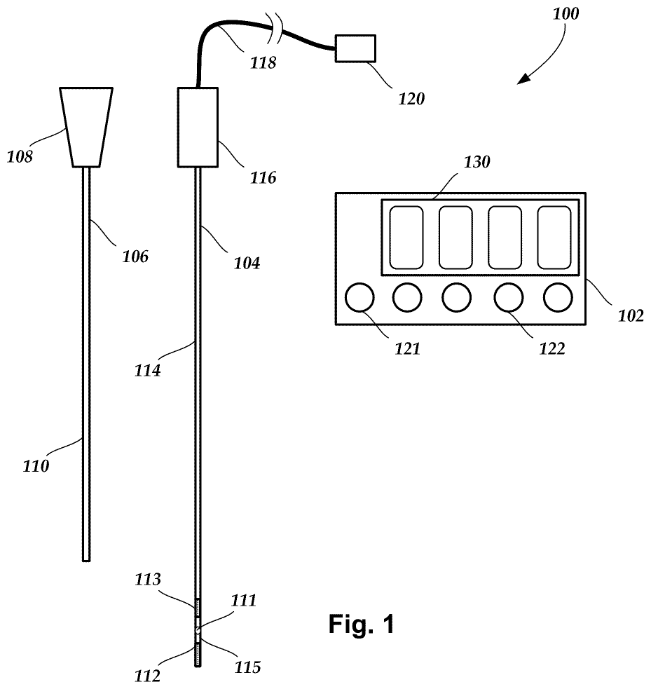

FIG. 1 illustrates one embodiment of a RF ablation system 100 that includes a RF generator 102, a bipolar RF electrode 104, and a cannula 106. It should be appreciated that a RF electrode may be a multipolar RF electrode having multiple electrodes on the same shaft. It will be recognized that some embodiments of a RF ablation system can include more or fewer components.

The cannula 106 includes a cannula hub 108 and a cannula shaft 110. The cannula shaft 110 is hollow for receiving the bipolar RF electrode 104. The bipolar RF electrode 104 includes an electrode shaft 114, a first electrode 112, a second electrode 113, an insulative material 115 (which may be part of the electrode shaft) separating the first and second electrodes, at least one fluid delivery port 111 defined in the insulative material or electrode shaft and disposed between the first and second electrodes, an electrode hub 116, a cable 118 that is electrically coupled to the electrode shaft 114, and a connector 120 for coupling to at least one port 122 of the RF generator 102 to energize the first electrode 112 or second electrode 113 (or both) via the cable 118 and connector 120.

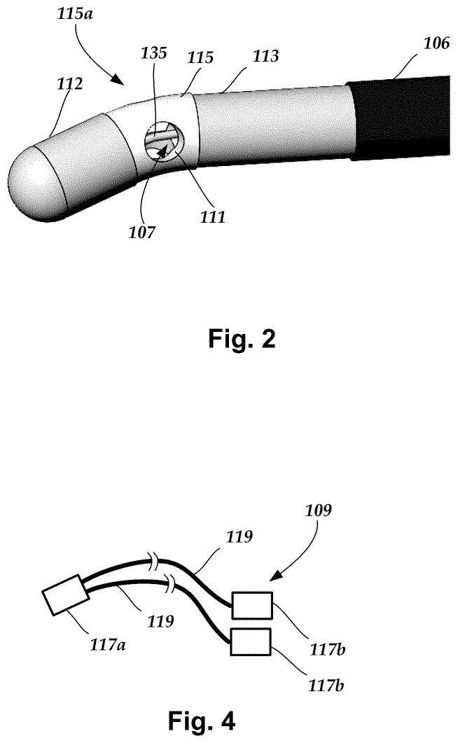

The electrode shaft 114 can be formed using one or more pieces. In at least some embodiments, the insulative material 115 is part of the electrode shaft 114. In at least some embodiments, the first and second electrodes 112, 113 are coupled to, or disposed along, one end portion of the electrode shaft 114 with the electrode hub coupled to, or disposed on, the opposite end portion of the electrode shaft. For example, the first and second electrodes 112, 113 can be attached, or disposed on, one end portion of the electrode shaft 114, as illustrated in FIGS. 1 and 2. In at least some embodiments, the electrodes 112, 113 are attached to the electrode shaft 114 using any suitable method including, but not limited to, adhesive attachment, attachment by reflow the material of the electrode shaft, attachment by injection molding to form at least a portion of the electrode shaft, or the like or any combination thereof.

The RF generator 102 can include one or more ports 122 and at least one screen 130. In at least some embodiments, each port 122 is associated with a portion of the screen 130 (or a different screen) and can receive the connector 120 from a bipolar RF electrode 104 or a connector from an adapter 109 (FIG. 4), as described below. Information such as current, voltage, impedance, status, or the like or any combination thereof can be displayed on the screen 130. In at least some embodiments, each port 122 corresponds to an independent channel. The RF generator 102 optionally includes a ground port 121.

Examples of RF generators and RF ablation systems and methods of making and using the RF generators and RF ablation systems can be found at, for example, U.S. Pat. Nos. 9,717,552; 9,956,032; 10,111,703; 10,136,937; 10,136,942; 10,136,943; 10,194,971; 10,342,606; 10,363,063; 10,588,687; 10,631,915; 10,639,098; and 10,639,101; U.S. Patent Application Publications Nos. 2014/0066917; 2014/081260; 2014/0121658; 2021/0121224; 2021/0236191; 2022/0202484; 2022/0202485; and 2022/0226039; U.S. patent application Ser. Nos. 17/553,555 and 17/574,400; and U.S. Provisional Patent Application Ser. Nos. 63/413,122 and 63/413,133, all of which are incorporated herein by reference in their entireties. At least some of these reference include examples of bipolar RF electrodes that can be modified to include at least one fluid delivery port between the electrodes, as described herein, as well as systems and methods that utilize the bipolar RF electrodes described herein.

FIG. 2 is a close-up view of distal ends of one embodiment of the bipolar RF electrode 104 and cannula 106 with the first electrode 112, second electrode 113, and one or more fluid delivery ports 111 in the insulative material 115 between the first and second electrodes. Any suitable number of fluid delivery ports 111 can be used including, but not limited to, one, two, three, four, or more fluid ports. In at least some embodiments, the bipolar RF electrode 104 includes multiple fluid delivery ports 111 disposed around the circumference of the bipolar RF electrode to direct fluid more uniformly to all of the tissue around the first and second electrodes 112, 113 than would be achieved using a single fluid delivery port.

In other embodiments, the fluid port(s) 111 are arranged to direct fluid in a preferential direction or directions. For example, a bipolar RF electrode 104 can have a single fluid port 111 that is intended to direct fluid toward the BVN when the first and second electrodes 112, 113 are positioned within the vertebra with the single fluid port 111 defined in the side of the insulative material 115 nearest the BVN.

In at least some embodiments, as illustrated in FIG. 2, the distal end of the bipolar RF electrode 104 can include a bend 115a. In the illustrated embodiment and at least some other embodiments, the bend 115a is formed in the insulative material 115 between the first and second electrodes 112, 113. In other embodiments, alternatively or additionally one (or both) of the electrodes 112, 113 include(s) a bend. The bend 115a can facilitate placement of the electrodes 112, 113 near the BVN in the vertebra. In at least some embodiments, the first electrode 112 is a tip electrode that is not open at the distal end, as illustrated in FIG. 2. In at least some embodiments, the first electrode 112 is a tip electrode that is capped or closed at the distal end.

Returning to FIG. 1, the bipolar RF electrode 104 has two conductors (such as conductor 135 illustrated in FIG. 2) that extend along the cable 118, optionally through the electrode shaft 114, and couple to the first and second electrodes 112, 113, respectively. In at least some embodiments, one conductor is electrically coupled to one of the electrodes (for example, electrode 112) and supplies power to that electrode and the other conductor is electrically coupled to the other one of the electrodes (for example, electrode 113) and acts as a return. In at least some embodiments, at least the electrode shaft 114, the second electrode 113, and the insulative material 115 have a hollow interior 107 to allow passage of the conductors 135, as well as fluid, through the bipolar RF electrode 104.

In at least some embodiments, the conductors 135 are insulated. In at least some embodiments, at least one (or both) of the first and second electrodes 112, 113 are insulated within the hollow interior 107 defined by at least the electrode shaft 114, the second electrode 113, and the insulative material 115. For example, the second electrode 113 can be disposed over a portion of a plastic electrode shaft 114 with an opening in the electrode shaft for passage of a conductor 135 and connection to the second electrode. The first electrode 112 can be inserted into the tip of the electrode shaft 114 or insulative material 115. (In at least some embodiments, the insulative material 115 may also be part of the electrode shaft 114.) Insulation of the conductors 135 and at least one of the first or second electrodes 112, 113 may reduce or prevent shorting of the first and second electrodes due to fluid residing in, or flowing through, the bipolar RF electrode 104.

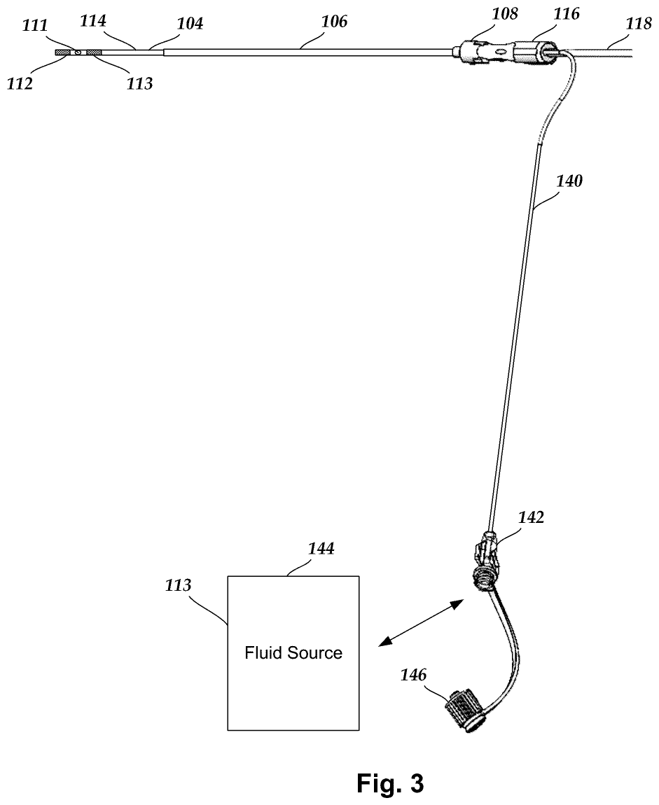

FIG. 3 illustrates one embodiment of the bipolar RF electrode 104 disposed in the cannula 106 and a fluid line 140 extending from the electrode hub 116 or electrode shaft 114 of the bipolar RF electrode. In at least some embodiments, the fluid line 140 is a flexible tubing. The fluid line 140 is in fluid communication with the interior 107 (FIG. 2) defined by at least the electrode shaft 114, the second electrode 113, and the insulative material 115. The fluid line 140 is attached to a connector 142, such as a Luer connector, that can be coupled to a fluid source 144, for example a syringe, for delivery of fluids through the fluid line 140, electrode shaft 114, and out the at least one fluid delivery port 111 between the first and second electrodes 112, 113. The connector 142 optionally includes a cap 146.

As one example of use, before ablation a clinician can deliver a numbing agent, such as lidocaine, through the at least one fluid delivery port 111 between the first and second electrodes 112, 113. A significant decrease in the patient's pain confirms that the bipolar RF electrode 104 is in the correct position to ablate the desired nerve, such as the BVN, or other tissue. The numbing agent can also relieve or reduce any discomfort or pain felt by the patient during ablation. Moreover, in at least some embodiments, the use of the numbing agent can confirm that the patient is a good candidate for ablation therapy.

In at least some embodiments, the numbing agent or other electrically conductive fluid, such as water, saline, or any other conductive fluid or fluid that becomes conductive when mixed with bodily fluids at the target site, delivered through the at least one fluid delivery port 111 can also increase the conductivity at the ablation site to ensure that the desired target nerve, such as the BVN, or other tissue is ablated. This can result in a larger ablation volume than if no fluid was delivered because RF energy needs a conductive medium to increase the temperature to ablate the tissue.

Ablation, such as BVN ablation, does cause harm to the target tissue, such as the BVN. The unablated nerve endings subsequently heal or repair themselves. In a least some embodiments, the clinician can inject healing medication, such as a steroid, through the at least one fluid delivery port 111 between the first and second electrodes 112, 113 to speed up the healing process. Faster healing may reduce the painful time interval after the ablation procedure.

Another use for the fluid port can be to inject a contrast agent through the at least one fluid delivery port 111 between the first and second electrodes 112, 113 to facilitate imaging and visualization of the target site using, for example, MRI, fluoroscopy, or the like or any combination thereof.

In at least some embodiments, embolic beads can be passed through the at least one fluid delivery port 111 between the first and second electrodes 112, 113 to starve the nerves or other target tissue of blood flow and effectively kill the nerve or other tissue, such as a tumor, as an alternative or supplement to ablation.

At least some RF generators provide a single channel at each port 122. In at least some embodiments, the bipolar RF electrode 104 uses a separate channel for each of the two electrodes 112, 113. In at least some embodiments, the RF ablation system 100 can include an adapter 109, illustrated in FIG. 4, with a connector 117a to connect to the connector 120 of the bipolar RF electrode 104, two cables 119 that are individually coupled through the connector 117a to a different one of the conductors (such as conductors 135 illustrated in FIG. 2) of the bipolar RF electrode, and two port connectors 117b for coupling to individual ports 122 of the RF generator 102. This permits one port 122 to energize one of the electrodes (for example, electrode 112) and another port 122 to act as a return using the other of the electrodes (for example, electrode 113.)

The above specification provides a description of the structure, manufacture, and use of the invention. Since many embodiments of the invention can be made without departing from the spirit and scope of the invention, the invention also resides in the claims hereinafter appended.

What is claimed as new and desired to be protected by Letters Patent of the United States is:

1. A bipolar RF electrode, comprising:
an electrode shaft having a first end portion and a second end portion opposite the first end portion;
a first electrode;
a second electrode attached to the second end portion of the electrode shaft;
an insulative material coupled to, and disposed between, the first electrode and the second electrode, the insulative material defining at least one fluid delivery port; and
an electrode hub attached to first end portion of the electrode shaft, wherein the electrode hub or the electrode shaft is configured for attachment of a fluid line, wherein at least the electrode shaft, the second electrode, and the insulative material form a hollow interior for flow of fluid from the fluid line, when attached, to the at least one fluid delivery port defined by the insulative material and disposed between the first electrode and the second electrode.

2. The bipolar RF electrode of claim 1, wherein the at least one fluid delivery port is a plurality of fluid delivery ports disposed around a circumference of the bipolar RF electrode.

3. The bipolar RF electrode of claim 1, wherein the electrode hub is configured for attachment of the fluid line.

4. The bipolar RF electrode of claim 1, wherein the electrode shaft is configured for attachment of the fluid line.

5. The bipolar RF electrode of claim 1, further comprising a cable extending from the electrode hub and a plurality of conductors extending along the cable and the electrode shaft, wherein at least one conductor is electrically coupled to the first electrode and at least one other conductor is electrically coupled to the second electrode.

6. The bipolar RF electrode of claim 1, wherein the first electrode has a closed end.

7. The bipolar RF electrode of claim 1, wherein the insulative material is part of the electrode shaft.

8. The bipolar RF electrode of claim 1, wherein the second electrode is disposed over a portion of the electrode shaft.

9. A kit, comprising:
the bipolar RF electrode of claim 1; and
a cannula configured for insertion of the electrode shaft through the cannula.

10. The kit of claim 9, further comprising the fluid line.

11. A RF ablation system, comprising:
the bipolar RF electrode of claim 1;
a cannula configured for insertion of the electrode shaft through the cannula; and
a RF generator configured for electrically coupling to the bipolar RF electrode and energizing at least one of the first electrode or the second electrode.

12. The RF ablation system of claim 11, further comprising the fluid line.

13. The RF ablation system of claim 12, further comprising a fluid source coupleable to the fluid line for providing fluid to the fluid line for delivery through the bipolar RF electrode and out the at least one fluid delivery port.

14. A method for performing RF ablation, the method comprising:
positioning the first and second electrodes of the bipolar RF electrode of claim 1 proximate to an ablation target of a patient;
delivering fluid from a fluid source through a fluid line and the bipolar RF electrode coupled to the fluid line and out the at least one fluid delivery port defined by the insulative material of the bipolar RF electrode; and
ablating tissue using the first and second electrodes of the bipolar RF electrode.

15. The method of claim 14, wherein the fluid comprises a numbing agent.

16. The method of claim 15, further comprising confirming the positioning of the first and second electrodes by numbing of the patient by the numbing agent.

17. The method of claim 14, wherein the fluid comprises a contrast agent, the method further comprising imaging the ablation target and first and second electrodes after delivery of the contrast agent.

18. The method of claim 14, wherein the fluid comprises a conductive liquid to enhance ablation.

19. The method of claim 14, wherein the fluid comprises a healing medication, wherein the delivering occurs after the ablating.

20. The method of claim 14, wherein the fluid comprises embolic beads configured to starve tissue of blood flow.
